SEQUENCE LISTING (10 sequences)

SEQ ID NO: 1 - protein, 9 aa, Artificial Sequence
  synthetic construct
EEQFNSTFR

SEQ ID NO: 2 - protein, 9 aa, Artificial Sequence
  synthetic construct
EEQFNSTYR

SEQ ID NO: 3 - protein, 10 aa, Artificial Sequence
  synthetic construct
LSVPTSEWQR

SEQ ID NO: 4 - protein, 14 aa, Artificial Sequence
  synthetic construct
FTCLATNDAGDSSK

SEQ ID NO: 5 - protein, 7 aa, Artificial Sequence
  synthetic construct
DTLMISR

SEQ ID NO: 6 - protein, 21 aa, Artificial Sequence
  synthetic construct
GYLPEPVTVTWNSGTLTNGVR

SEQ ID NO: 7 - protein, 22 aa, Artificial Sequence
  synthetic construct
SVSLTCMINGFYPSDISVEWEK

SEQ ID NO: 8 - protein, 25 aa, Artificial Sequence
  synthetic construct
QSSGLYSLSSVVSVTSSSQPVTCNV

SEQ ID NO: 9 - protein, 14 aa, Artificial Sequence
  synthetic construct
VYTMGPPREELSSR

SEQ ID NO: 10 - protein, 6 aa, Artificial Sequence
  synthetic construct
TFPSVR

(19) United States
(12) Patent Application Publication
(10) Pub. No.: US 2013/0195873 A1
(43) Pub. Date: Aug. 1, 2013

(54) CARBOHYDRATE HAPTEN-BASED ANTI-CANCER VACCINES AND ANTIBODY DRUGS

(71) Applicant: Chi-Yu Gregory Lee, Vancouver (CA)

(72) Inventor: Chi-Yu Gregory Lee, Vancouver (CA)

(73) Assignee: Vancouver Biotech LTD., Vancouver, BC (CA)

(21) Appl. No.: 13/810,199

(22) PCT Filed: Jul. 14, 2011
(86) PCT No.: PCT/CA2011/000802

§ 371 Date: Mar. 26, 2013

Related U.S. Application Data
(60) Provisional application No. 61/364,319, filed on Jul. 14, 2010.

(51) Int. Cl. C07H 13/04; A61K 39/00

(52) U.S. Cl. CPC C07H 13/04; A61K 39/0011
USPC 424/137.1; 536/53; 530/395; 424/193.1; 424/277.1; 530/387.5; 435/69.6

(57) ABSTRACT

Immunogenic compositions that contain haptens consisting of carbohydrate moieties are useful to induce an immune response to provide antibodies to epitopes contained in CA215 and also to elicit an immune response to cancers expressing these epitopes.

### CROSS-REFERENCE TO RELATED APPLICATION

This application claims priority from U.S. provisional application 61/364,319 filed 14 Jul. 2010. The contents of this document are incorporated herein by reference.

### TECHNICAL FIELD

The invention is in the field of cancer immunology. More specifically it is directed to carbohydrate moieties that may serve as the hapten components of anticancer vaccines.

### BACKGROUND ART

U.S. Pat. No. 5,650,951 describes and claims RP215, a monoclonal antibody that immunoreacts with CA215 a marker which is present on many cancers. RP215 induces apoptosis and complement-dependent cytotoxicity in different cancer cell cultures in vitro (Lee, G., and Ge, B., Cancer Immunol. Immunother. (2010) 59:1347-1356) and also inhibits tumor growth in nude mouse models. Lee, G., et al., Cancer Biol. & Ther. (2009) 8:161-166.

In addition, rat monoclonal antibodies generated against an antiidiotypic antibody to RP215 induce significant Ab3 responses in mice, and these, too, are shown to induce apoptosis in cultured cancer cells (Lee and Ge, supra). RP215 has also been used to formulate immunoassay kits to monitor serum levels of CA215.

PCT publication WO2008/138139 describes features of an effective epitope of this pan cancer marker, CA215, that is recognized by the RP215 monoclonal antibody. The CA215 antigen itself has immunoglobulin-like characteristics. It was disclosed in this publication that the epitope per se includes at least a carbohydrate moiety coupled to the variable region of the Ig-like heavy chain, but the epitope itself does not bind anti-human Ig, and this document includes a detailed description of N-linked glycans associated with this antigen and a generic description of the O-linked glycans as a rough analysis.

It has now been found that the epitope recognized by RP215 consists of one or more carbohydrates unique to CA215, as opposed to glycans found on immunoglobulins generally. In addition, the structural features of the O-linked glycans have been elucidated such that the relevant haptens can be synthesized de novo and manipulated to provide defined compositions.

### DISCLOSURE OF THE INVENTION

The components found in the O-linked and N-linked glycans in CA215 include acetylgalactosamine (GalNAc), N-acetylglucosamine (GlcNAc), N-acetylneuraminic acid (NeuAc), N-glycolylneuraminic acid (NeuGc), galactose (Gal), mannose (Man) and fucose (Fuc). Structures are shown in FIG. 1.

In one aspect, the invention is directed to an immunogenic composition that contains as an immunogen at least one hapten consisting of a carbohydrate selected from the group consisting of:

GalNAc₁GlcNAc₁Gal₁NeuAc₁;

GalNAc₁Gal₁NeuAc₂;

GalNAc₁Gal₁NeuGc₂;

GalNAc₁GlcNAc₁Gal₂NeuAc₁; and

GalNAc₁GlcNAc₁Gal₂NeuGc₁;

wherein said composition further comprises an adjuvant, and/or wherein one or more of said haptens are coupled to a heterologous protein.

The foregoing tetra- and penta-saccharides or glycans are themselves novel. Thus, the invention is also directed to these glycans themselves as compounds and intermediates.

The invention also includes compositions that comprise at least one of the oligosaccharides set forth above along with an oligosaccharide that is an N-linked glycan associated with CA215 selected from the group consisting of

GlcNAc₅Man₃Hex₂;

GlcNAc₂Man₃;

GlcNAc₄Man₃Hex₂Fuc₁NeuGc₁;

GlcNAc₅Man₃Hex₂NeuGc₁;

GlcNAc₄Man₃Hex₂NeuGc₂;

GlcNAc₅Man₃Hex₃NeuGc₁;

GlcNAc₆Man₃Hex₄;

GlcNAc₄Man₃Hex₂Fuc₁NeuGc₂;

GlcNAc₂Man₅;

GlcNAc₃Man₃Hex₁; and

GlcNAc₂Man₆.

The compositions may supply the carbohydrate moieties as separate entities or coupled using linkers or backbones of various types, such as peptide or polyethylene glycol linkers. As noted previously, PCT publication WO2008/138139 places the carbohydrate epitope on the variable region of the immunoglobulin-like chain included in CA215. By using the positions of the glycans as revealed by the O-linked, and possibly N-linked glycosylation sites in this region, epitopes that involve more than one glycan moiety may be designed using alternative backbone moieties.

In other aspects, the invention is directed to methods to elicit an immune response directed to a cancer expressing a glycan epitope of the invention in a subject by administering the compositions of the invention. In still another aspect, the invention is directed to methods to prepare antibodies immunospecific for these epitopes by administering the immunogenic compositions of the invention, and to these antibodies per se.

### BRIEF DESCRIPTION OF THE DRAWINGS

FIG. 1 shows the structures of GalNAc, GlcNAc, NeuAc, NeuGc, Gal, Man and Fuc. Particular enantiomers of the carbohydrates are shown; the designations herein are intended to refer to either enantiomer. Thus, the structure of L-fucose is illustrated, but D-fucose is also included within the scope of the invention.

FIGS. 2A and 2B show NSI-MS spectra of permethylated O-linked glycans from (A) CA215 (lot CA215C) and (B) RP215. The symbol “*” in FIGS. 1A and 1B, indicates the N-linked glycan released by O-elimination. The assigned glycan structures are indicated inside FIG. 1A with mass and charge (z) indicated.

FIG. 3 is a gas chromatogram of partially methylated alditol acetates (PMAAs) of the O-linked glycans of CA215 from urea-eluted CA215 (lot#S15K-100425). The elution time (in min), of which the linkage relationship is given in each carbohydrate element, and the positions of linked glycans are indicated inside the profiling diagram by A (terminal Gal), B (3-linked Gal), C (3-linked GalNAc reduced), D (3,6-linked GalNAc reduced) and E (4-linked GlcNAc).

FIGS. 4A and 4B illustrate the MALDI-TOF MS spectra of permethylated N-glycans from CA215 and a gas chromatogram of partially methylated alditol acetates of CA215 N-glycans.

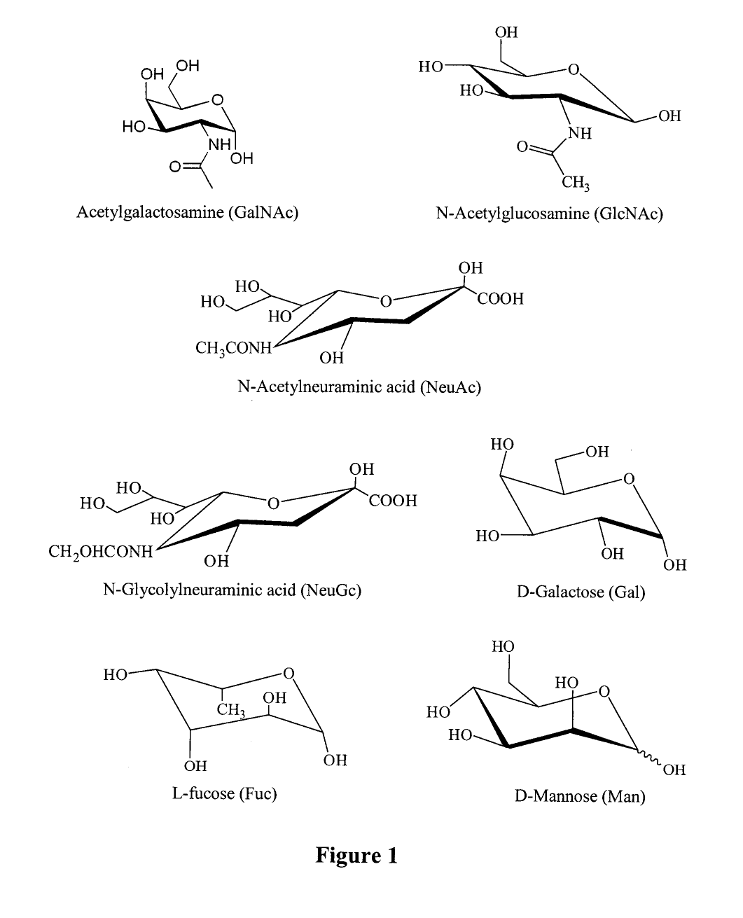

### MODES OF CARRYING OUT THE INVENTION

The identification of specific carbohydrate moieties as the epitopes with which CA215 immunoreacts with RP215 represents an opportunity to synthesize, directly, anticancer vaccines with defined components. The examples below show the structures of the relevant glycans. Methods for synthesis of such glycans are well known in the art. Thus, each of the five O-linked glycans of the invention may readily be synthesized using methods known in the art.

The synthetic glycans may not be sufficiently immunogenic per se without appropriate linking to defined synthetic peptides in order successfully to function as vaccines or to generate antibodies. Accordingly, the invention contemplates compositions which contain adjuvants to enhance immunogenicity and/or conjugates of the carbohydrate moieties to defined synthetic peptides and/or to heterologous proteins to enhance immunogenicity. Available heterologous proteins for this purpose are well known, including keyhole limpet hemocyanin (KLH), bovine serum albumin (BSA) and tetanus toxoid. Suitable adjuvants include aluminum salts such as aluminum phosphate and aluminum hydroxide, liposomes, polymers such as polylactide-co-glycolide microspheres, muramyl dipeptide (MDP), monophosphoryl lipid A (MPLA) and CpG oligodeoxynucleotide (ODN). The nature of adjuvants and the many adjuvants available are well known.

The complete epitope may contain more than one of the relevant glycans, and a suitable backbone may be employed to couple one or more of the glycans described below to a common matrix. Alternatively, mixtures of the relevant glycans may be employed. Various carriers, such as liposomes, nanoparticles, and the like may also be used.

In addition to the O-linked glycans whose structures have been set forth below, the epitopes may further include one or more N-linked glycans unique to CA215 as set forth above. Again, the N-linked and O-linked derived glycans may be coupled to a backbone for presentation to the immune system. They may also be coupled to adjuvants or adjuvants may simply be added to the composition.

The compositions of the invention which include carbohydrates and optionally synthetic peptides, heterologous proteins and/or adjuvants are useful as vaccines to induce an immune response to cancers in subjects wherein the cancers express epitopes set forth above. Animal model subjects, such as mice, rats, rabbits, guinea pigs, and the like, may be administered such vaccines to optimize the formulation and protocols. Human subjects may be treated with additional therapies such as radiation and chemotherapy along with the immunogenic compositions of the invention.

Immunohistochemical staining studies of normal and cancerous tissues have demonstrated that the epitope recognized by RP215 is present on a number of types of human cancers, with varying levels of staining intensity. The epitope shows very intense staining on human cancers of the ovary, cervix, endometrium, colon, stomach, intestine, esophagus, breast, and lung. As noted herein, the tumor tissues from any particular subject can be evaluated using immunostaining for the presence and level of this epitope, thus providing information useful in the design of suitable vaccines, whether composed of the epitope itself or an antiidiotype antibody that mimics it as further described below.

Suitable formulations for the defined epitope of the invention are those conventional for immunogenic compositions and are found, for example, in Remington's Pharmaceutical Sciences, latest edition, Mack Publishing Co., Easton, Pa., incorporated herein by reference. Protocols for administration are dependent on the nature of the condition, the judgment of the attending physician, and the severity of the malignancy. Optimization of such protocols on a group or individual basis is well within ordinary skill.

Modes of administering these compositions are according to standard procedures and the judgment of the physician; typical administration is by injection, including intravenous, intramuscular and subcutaneous injection. However, other modes of administration may be devised including other parenteral methods as well as oral administration.

The compositions of the invention are also useful in the preparation of additional antibody compositions, including monoclonal antibody compositions that can be used in a manner analogous to RP215 in standard assay procedures as well as for passive immunization. As used herein, “antibodies” includes complete immunoglobulins as well as immunospecific fragments thereof, such as Fab, Fab_{2′} and Fᵥ fragments. The antibodies may be monoclonal, prepared by standard and well known techniques and under these circumstances may be manipulated recombinantly to obtain humanized forms, chimeric forms in which the variable region associated with one species is coupled to a constant region associated with another or may be single-chain antibodies. Techniques for manipulation of monoclonal antibodies using the tools of recombinant production are well established. The epitopes of the invention may also be used as purification and identification tools for suitable antibodies. The compositions of the invention may be used to elicit an immune response in a suitable subject, such as an laboratory animal, e.g., a rodent, rabbit, or other suitable non-human subject to obtain polyclonal preparations useful in assay procedures.

Alternatively, antibody-producing cells may be isolated from the immunized subject, and immortalized and screened for the production of monoclonal antibodies. These monoclonal antibodies may, if desired, be prepared recombinantly by isolating the nucleic acids encoding them from the antibody-producing cells and manipulating the nucleic acids for recombinant production. Accordingly, modified forms of the antibodies, such as single-chain or Fv antibodies may be produced.

For use in passive immunization of humans, the recombinantly produced antibodies may also be humanized using standard techniques or they may be produced by immunizing suitable subjects such as the XenoMouse® thus directly producing human antibodies by immunization with the glycan-containing compositions of the invention.

Synthesis of a CA215 epitope that is immunogenic is universally useful, as the epitope recognized by RP215 is shared by CA215 of various cellular origins in human cancers. The Examples below show that apparent affinity between RP215 and CA215 antigen derived from different sources does not differ significantly, with Kd's in the range of 2-4 nM, suggesting that the RP215-specific epitope is unique in carbohydrate structure and does not vary significantly with the associated glycopeptides derived from different cancer cells or origins.

The glycosylation site mapping described below shows both the detected N-linked and O-linked glycopeptides were matched predominantly to immunoglobulin heavy chains, so that both N-linked and O-linked glycans may be involved in epitope recognition by RP215; in particular the glycan structure with terminal NeuGc is found in both types of glycans with mucin-like structures, though recent data suggest NeuGc may not be critical for CA215 binding to RP215.

Sandwich EIA described in the Examples shows that RP215 recognizes preferentially the carbohydrate-associated epitope in the Fab regions of immunoglobulin heavy chains of CA215, rather than the Fc region. However, the Examples below show that detected N-linked glycopeptides have 100% homology to those of immunoglobulin heavy chain Fc regions. One of the O-linked glycopeptides, FTCLATNDAGDSSK, has 100% homology to IgSF proteins, including hemicentin (Vogel, B. E., and Hedgecock, E. M., Development (2001) 128:883-894) and titin (Labeit, S., and Kolmerer, B., Science. (1995) 270:293-296). Surprisingly, another O-linked glycopeptide, LSVPTSEWQR, has a high degree of homology to cathepsin S which is a known lysosomal protease (Shi, G. P., et al., J. Biol. Chem. (1992) 267:7258-7262). Unexpectedly, the remaining O-linked glycopeptides were all matched to immunoglobulin heavy chains.

From the results of glycan profiles presented in this study, significant differences in the N-linked and O-linked glycans have been shown between CA215 and normal human IgG. For example, the N-glycans of CA215 have high mannose content and include NeuGc, but these are not found in normal human IgG.

The instability of RP215-specific immunoactivity in CA215 observed at extreme pH (≦2.0 and ≧12.0) suggests that RP215-specific epitope may include one or more O-glycans, possibly containing terminal NeuGc, and while N-linked glycans may be less important, since the incubation of cultured cancer cells with tunicamycin had little effect on the CA215 immunoactivity, these N-linked glycans may participate as well, because the RP215-specific epitope may involve tetra- and/or penta-oligosaccharides with terminal NeuGc identified in the O-glycan or N-glycan profiling analysis of CA215.

Analyses of tryptic peptides described in the Examples from CA215 and the evaluation peptide sequence homology show that the majority of tryptic peptides of CA215 match those of immunoglobulin superfamily (IgSF) proteins and mucins, suggesting common domain structures in IgSF proteins that could result in the preferential glycosylation of this group of glycoproteins (Barclay, A. N., Seminars in Immunology (2003) 15:215-223). Mucins are heavily glycosylated glycoproteins in normal or cancer cells, and RP215-specific epitope(s) are also generated in mucins expressed preferentially by cancer cells.

The glycan structures of mucins in the normal and cancerous tissues have been analyzed previously (Yamashita, K., et al., Cancer Research (1995) 55:1675-1679; Podolsky, D. K., et al., J. Biol. Chem. (1985) 260:8262-8271; Podolsky, D. K., et al., J. Biol. Chem. (1985) 260:15510-15515). The terminal NeuAc of α2-6 linkages is preferentially identified in the normal tissue, but, α2-3 linkage of terminal NeuAc or NeuGc is predominantly found in the cancerous tissue. Also, the linkages between Gal and GlcNAc (β-1,3 versus β-1,4) were different in the normal versus cancerous tissue (Qiu, X., et al., Cancer Research (2003) 63:6488-6495; Kurosaka, A., et al., J. Biol. Chem. (1983) 258:11594-11598; Capon, C., et al., J. Biol. Chem. (1992) 267:19248-19259; Malykh, Y. N., et al., Biochimie. (2001) 83:623-634). The differential sialic acid linkages as well as the appearance of NeuGc result in the creation of new RP215-specific epitope(s).

Our previous studies have shown that CA215 with RP215-specific carbohydrate-associated epitope was widely expressed by almost all of the cancer tissues in humans. Abnormal glycosylation in cancer cells results in the formation of a new immunogenic carbohydrate moiety which has never been identified previously in immunoglobulins of normal B cell origins, but can be recognized by RP215 Mab. Previous studies by others also indicated that immunoglobulins expressed by cancer cells and other normal human cells of non-B cell origins might be important for the growth promotion of cancer cells as well as others in vivo (Qiu, et al. (supra, 2003)).

As noted above, it is clear that at least one O-linked glycan as set forth in Table 1 is present in the epitope responsible for the binding of CA215 to RP215. It is also possible that one or more N-linked glycans is included. Therefore, compositions of the invention include those which contain multiple O-linked glycans of Table 1 or at least one O-linked glycan from Table 1 and one or more N-linked glycans as set forth in Table 3 below. While simple mixtures of these glycans may be used when more than one glycan is present, it is preferable to link the glycans in the composition by means of binding them to a backbone, such as a peptide backbone or other polymeric support. Bifunctional linking agents may also be used.

As it is shown in the examples below that the epitope appears to be present predominantly on the Fab region of the immunoglobulin light chain in CA215, peptides from the Fab region of this chain are preferred backbone moieties. The N- and O-linked glycosylation sites on this region of the immunoglobulin-like portion of CA215 are conveniently used. The sequences of peptides derived from the variable (Fab) region of the CA215 from cells of various origins were described in the above cited WO2008/138139 publication. Using the peptides and sites of glycosylation described in this publication as a guide as well as the sequences of the peptides containing them, one may readily design alternative backbone structures that mimic the three-dimensional conformation of the native epitope. The disclosure of such sequences and sites derived from various CA215 antigens as set forth in this publication are incorporated herein by reference.

Thus, synthetic peptides that contain suitable glycosylation sites may be coupled to the carbohydrate moieties of the invention using standard synthetic methods. Alternatively, pseudopeptides containing, for example, CH₂NH₂ linkages in place of peptide linkages may be substituted or alternative oligomers comprising functional groups capable of binding glycans can be used. The skilled artisan can readily use the glycopeptide residues described in the above noted publication as a guide to design a mimicking epitope.

The following examples are offered to illustrate but not to limit the invention.

### Example 1

### Confirmation of O-Linked Glycan Epitope Relevance

It has been shown that treatment of CA215 with pH levels either below 2 or above 12 reduces the immunoreactivity of CA215 with RP215. As this treatment is destructive of O-linked glycosylation, these results indicate the involvement of O-linked glycans in the immunoreactivity of CA215. Treatment of cell cultures expressing CA215 with tunicamycin, which inhibits N-glycosylation, does not result in production of CA215 with reduced immunoreactivity. It thus appears that N-linked glycosylation is less relevant to the composition of the epitope. However, both N-linked and O-linked glycopeptides were matched, as shown in the Examples below, to immunoglobulin heavy chains. Thus, N-linked saccharides may also be participants in the epitope. Especially suitable glycans are those with terminal NeuGc or NeuAc residues. It is thus demonstrated that the relevant carbohydrate epitope is one or more O-linked glycans and/or may include N-linked glycans preferably with terminal NeuGc or NeuAc residues.

### Example 2

### Determination of Carbohydrate Epitopes

CA215 from the spent culture medium of OC-3-VGH cells (Lee, B. Y. G., et al., Cancer Immunol. Immunother. (1992) 35:19-26) was isolated by RP215-affinity chromatography as described in Lee, G., et al., Cancer Biology and Therapy (2008) 7:91-98. Briefly, 200 ml of shed medium was passed through an RP215 affinity column. After extensive wash with PBS, the bound CA215 was eluted either with 5 mM citric acid at pH 2.5 or with 3M urea. Fractions containing optical absorbance at 280 nm were collected and dialyzed.

The immunoactivity of affinity-purified CA215 was assayed by RP215-based sandwich EIA and purity was analyzed by SDS-PAGE and Western blot assay. Three different lots of purified CA215 were prepared and dialyzed separately against 10 mM NH₄HCO₃ and freeze-dried as the salt free form for glycoanalysis. Similarly, two lots of CA215 were also isolated from C-33A cultured shed medium and affinity-purified by the same method.

For parallel comparisons, purified normal human IgG and RP215 monoclonal antibody were prepared and subjected to similar analysis.

N-glycans were first released and removed, and the residue was subjected to β-elimination to release the O-linked glycans. The released O-glycans were desalted and cleaned of borate, permethylated and analyzed either by Nano Spray Ionization-Linear Ion trap Mass Spectrometry (NSI-LTQ™/MSn) or by Matrix-Assisted Laser Desorption Time-of-Flight Mass Spectrometry (MALDI-TOF MS).

To release N-linked glycans, the dried samples were dissolved in 0.1 M Tris-HCl buffer (pH 8.2 containing 10 mM CaCl₂), denatured by heating for 5 minutes at 100° C., cooled, and digested with trypsin at 37° C. overnight. After heating at 100° C. for 5 minutes to de-activate trypsin, PNGaseF (New England Biolabs) was added to release the N-glycans. The samples were then passed through a C18 reverse phase cartridge, and the N-linked glycans were first eluted with 5% acetic acid. The O-linked glycopeptides and peptides were eluted in series with 20% isopropanol in 5% acetic acid and 40% isopropanol in 5% acetic acid and then 100% isopropanol into separate fractions. The isopropanol fractions containing glycopeptide were evaporated initially under a steam of nitrogen and then lyophilized.

O-linked carbohydrates were cleaved from the glycopeptides by β-elimination. Briefly, 250 μL of 50 mM NaOH were added to each of the samples and verified for basic pH. An additional 250 μL of 50 mM NaOH containing 19 mg of sodium borohydride was added to the samples, vortexed, and incubated overnight at 45° C. The incubated samples were then neutralized with 10% acetic acid and desalted by passing through a packed column of Dowex® (Dow Chemical Co.) resins followed by lyophilization. Dried samples were cleaned of borate with methanol: acetic acid (9:1) under a stream of nitrogen gas before permethylation.

This was followed by per-O-methylation of the carbohydrates and purification thereof. The released O-glycans from each sample were dissolved in dimethyl sulfoxide and then methylated with NaOH and methyl iodide. The reaction was quenched by addition of water and per-O-methylated carbohydrates were extracted with dichloromethane. Per-O-methylated glycans were further cleaned of contaminants by passing through into a C18 Sep-Pak® (Waters Corporation, 34 Maple Street, Milford, Mass. 01757) cartridge and then washed with nanopure water. The glycans were then eluted with 85% acetonitrile. Purified glycans were dried under a stream of nitrogen gas and dissolved with methanol prior to analysis by mass spectrometry using nano spray ionization—linear ion trap-mass spectrometry (NSI-LTQ™-MSn) or matrix-assisted laser desorption/ionization time of flight mass spectrometry (MALDI-TOF MS).

For NSI-LTQ™/Msn, permethylated glycans were dissolved in 1 mM NaOH in 50% methanol and infused directly into the instruments (LTQ™, Thermo Finnigan™) at a constant flow rate of 0.4 μL/min. The capillary temperature was set at 210° C. and MS analysis was performed in the positive ion mode. Total ion mapping, which is an automated MS/MS analysis of all possible ions was performed at 2-mass unit interval from m/z 500 to 2000. Scanning was accomplished in successive 2.8 mass unit window with a collision energy of 28.

For MALDI-TOF MS, the permethylated glycans were dissolved with methanol and crystallized with α-dihydroxybenzoic acid. (DHBA 20 mg/ml in 50% methanol:water) matrix. Analysis of glycans present in the samples was performed in the positive ion mode by MALDI-TOF MS using Bruker microflex.

The results of the analyses of the O-linked glycans are shown in Table 1.

TABLE 1
Profiles of Permethylated O-linked Glycans of Human IgG and Five Different CA215 Samples

| | Observed Mass | | |
|---|---|---|---|
| Sample ID | m/z [M + Na]⁺ | Proposed Structure | Structure |
| CA215 (lots: A, B, and C)^{a} | 534 | GalNAc₁Gal₁ | 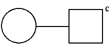 |
| CA215 (lots: A and B) | 708 | GalNAc₁Gal₁Fuc₁ | 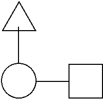 |
| CA215 (lots: A, B, D, C, E, and F)^{b} | 896 | GalNAc₁Gal₁NeuAc₁ | 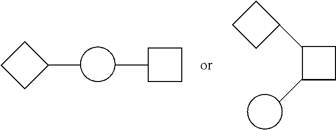 |
| CA215 (lots: C, E, and F) | 926 | GalNAc₁Gal₁NeuGc₁ | 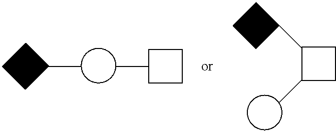 |
| CA215C | 940^{d} | GalNAc₁GlcNAc₁NeuAc₁ | 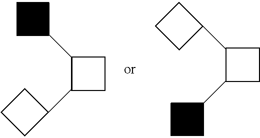 |
| CA215 (lots: A, B, and C) | 1140 | GalNAc₁GlcNAc₁Gal₁NeuAc₁ | 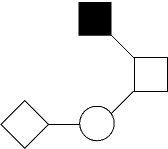 |
| CA215 (lots: C, D, E, and F) | 1257 | GalNAc₁Gal₁NeuAc₂ | 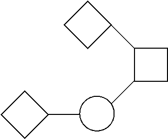 |
| CA215 (lots: C, E, and F) | 1317 | GalNAc₁Gal₁NeuGc₂ | 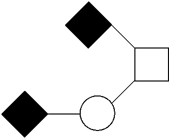 |
| CA215 (lots: A, B, C, E, and F) | 1345 | GalNAc₁GlcNAc₁Gal₂NeuAc₁ | 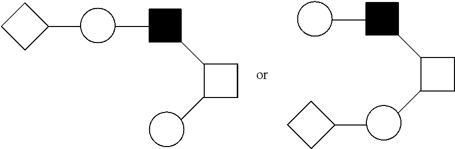 |
| CA215 (lots: C, E, and F) | 1375 | GalNAc₁GlcNAc₁Gal₂NeuGc₁ | 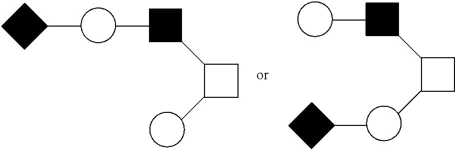 |

^{a}CA215 lots A, B and C were from OC-3-VGH ovarian cancer cells (CA215-OC-3) lots A and B were obtained through acid elution, whereas lots C, D, E and F were obtained through elution with 3M urea.
^{b} Lot CA215D was obtained by an additional purification of urea-eluted CA215 (S15K-100425) with goat anti-human IgG affinity column followed by the same analysis. CA215 lots E and F were from C-33A cervical cancer cells (CA215-C33A).
^{c}N-acetylgalactosamine (□), N-acetylglucosamine (▪), Fucose (Δ), Galactose (◯), N-acetylneuraminic acid (⋄), and N-glycolylneuraminic acid (♦)
^{d}Detected by MALDI-TOF MS method but not found by NSI-MS method

For the results in Table 1, a total of five different lots of purified CA215 from OC-3-VGH cells were employed in triplicate analyses. Two were obtained through purification by acid elution as described above (CA215A and CA215B), which reduced immunoactivity significantly. Therefore sample 215C was eluted from the immunoaffinity column with 3M urea. In addition, two urea-eluted lots of CA215 from the shed medium of C-33A cervical cancer cells were analyzed.

For NSI-LTQ™/MSn, CA215A and CA215B, were subjected to duplicate analyses, and both samples yielded identical results in terms of observed mass, charge state and proposed structure. Five different O-linked structures or fragments were identified and listed with masses ranging from 534 to 1346. They were identified as GalNAc₁Gal₁, GalNAc₁Gal₁Fuc₁, GalNAc₁Gal₁NeuAc₁, GalNAc₁GlcNAc₁Gal₁NeuAc₁, and HexNAc₂Gal₂NeuAc₁, respectively. NeuAc was the only terminal sialic acid observed among these O-linked fragments.

In a separate analysis with urea-eluted sample (lot CA215C), MALDI-TOF MS was employed. As many as ten different O-linked glycan structures or fragments were detected and also listed in Table 1. The mass observed ranged from 534 to 1375. Both NeuGc and NeuNAc were found among these O-glycan structures. Four of the detected O-linked fragments were identical to those observed by NSI-LTQ™/MSn method as described above, but GalNAc₁Gal₁Fuc₁ was not detected in this latter analysis. The remaining five O-glycans were detected only from the analysis by MALDI-TOF MS. Their primary structures are GalNAc₁Gal₁NeuGc₁, GalNAc₁NeuAc₁NeuGc₁, GalNAc₁Gal₁NeuAc₂, GalNAc₁Gal₁NeuGc₂ and HexNAc₂Gal₂NeuGc₁.

In a separate experiment, O-linked glycans analysis was also performed with a new lot of CA215 (lot CA215D) which was shown to be cancer cell-expressed human IgG through extensive biochemical and immunological analysis below. CA215D was obtained by affinity purification of urea-eluted CA215 (lot S15K-100425 from OC-3-VGH cancer cells) with goat anti-human IgG column. When subject to analysis by MALDI-TOF MS, only two major O-linked glycans were detected with terminal NeuAc. They are Gal₁NAc₁Gal₁NeuAc₁ (m/z 859.6) and GalNAc₁GalNAc₁NeuAc₂ (m/z 1256.9), respectively. Unexpectedly, other glycans were not detected in CA215D when compared with those of other singly-purified CA215 (lot CA215C).

By SDS-PAGE, CA215D was found to consist of mainly heavy and light chains with a molecular weight similar to those of normal human IgG (55 kDa and 25 kDa, respectively). By enzyme immunoassay (EIA) with goat anti-human IgG for both capturing and signal detection, both human IgG and CA215D revealed similar dose-dependent curves. Furthermore, by Western blot assay, both goat anti-human IgG and RP215 were shown to react with CA215D with a protein band of 60 kDa which is typical of the heavy chain of human IgG. However, RP215 does not react with normal human IgG by the same assay due to the absence of RP215-specific epitope.

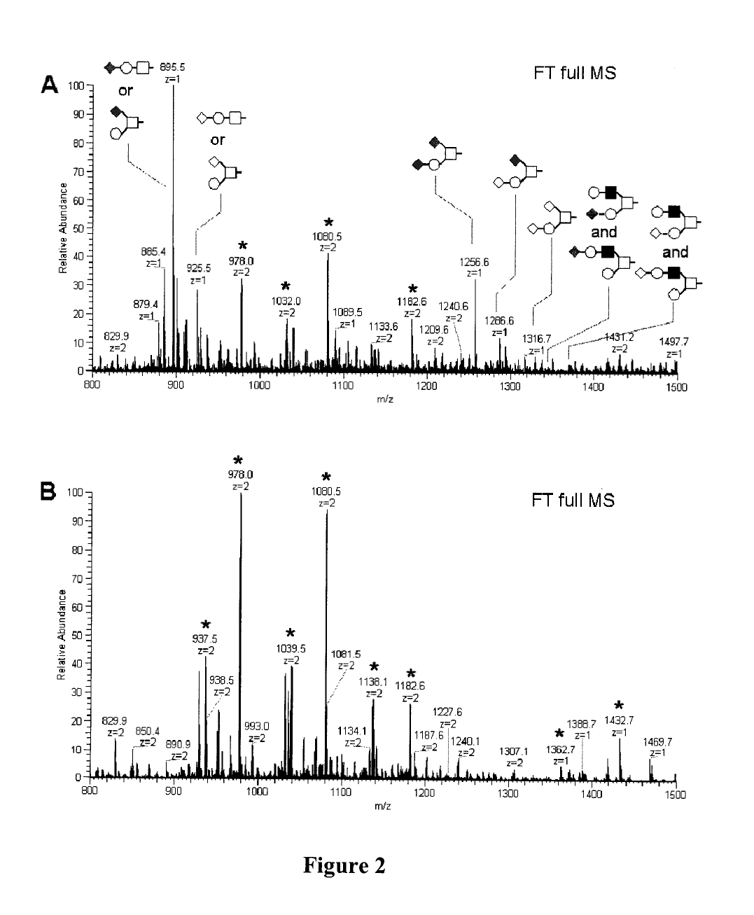

In a separate analysis with two urea-eluted lots of CA215 from the shed medium of C-33A cervical cancer cells, the O-glycan profile was found to be similar to that of CA215 from ovarian cancer cells. Typical NSI-MS spectra are presented in FIG. 2A. In contrast, the O-linked glycan profile was also analyzed with two lots of RP215 (FIG. 2B). Unexpectedly, no significant O-linked glycans were detected.

### Example 3

### Structural Determination of O-Linked Glycans

Linked Glycosyl Composition Analysis

Experiments were performed to analyze linkage relationships among different O-linked glycans, which are unique to CA215, but not detected in normal human IgG. They are briefly described as follows:

Partially methylated alditol acetates (PMAAs) were prepared from the permethylated O-linked glycans of CA215. Briefly, permethylated glycans were hydrolyzed with 2N trifluoroacetic acid at 121° C. for 2 hr, followed by reduction with NaBD₄. After hydrolysis and reduction steps, the free hydroxyls of the partially methylated alditols were acetylated with acetic anhydride: pyridine (1:1 v/v) at 100° C. for 1 hr to produce PMAAs, which were extracted with methylene chloride.

The PMAAs were analyzed on a Hewlett Packard 589° GC interfaced to 5970 MSD (Mass selective detector, electron impact ionization mode). The separation was performed on a 30 m EC1 bounded phase fused silica capillary column (Altech). Electron impact mass spectra were obtained under the following conditions: oven temperature, 140° C. (2.0° C./min)→220° C. (20° C./min)→300° C. (7.5 min); detector temperature, 280° C., inlet temperature, 250° C.

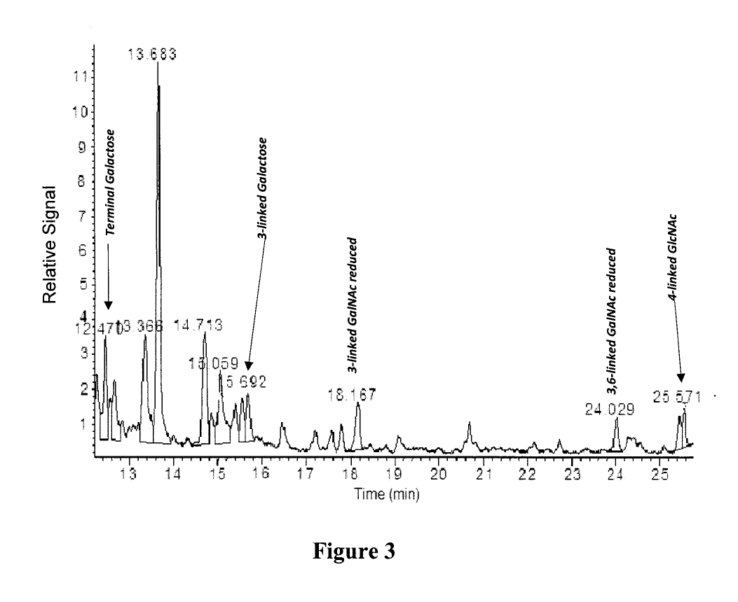

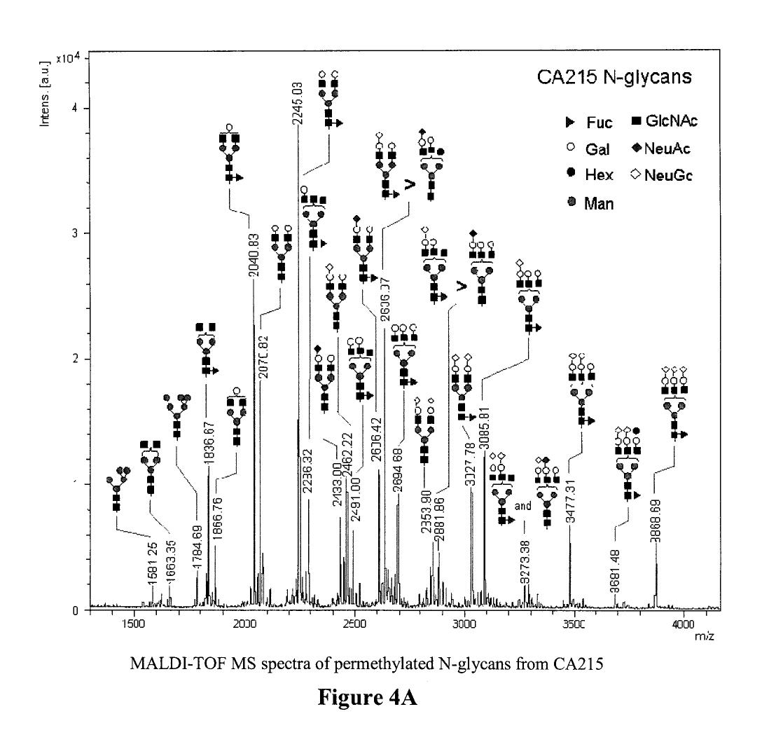

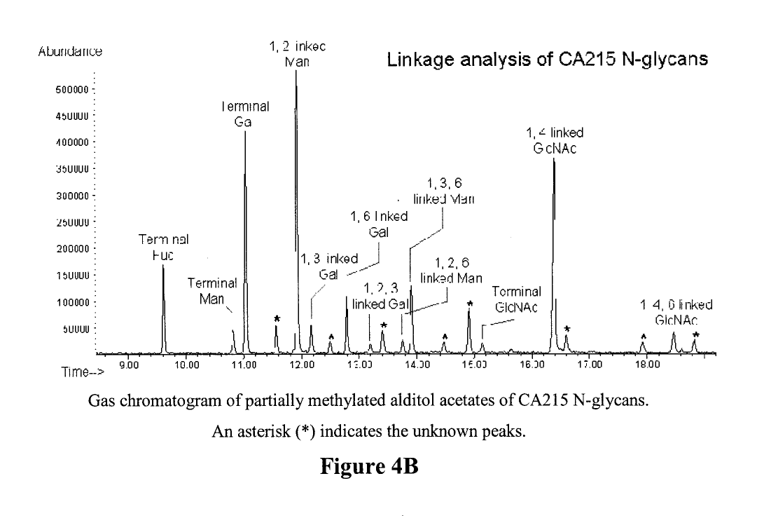

The following linked monosaccharides were detected in CA215 lot #515K-S10045 as shown in FIG. 3:

terminal galactose (time 12.470 min),

3-linked galactose (time 15.692 min),

3-linked GalNAc reduced (time 18.167 min),

3,6-linked GalNAc reduced (time 24.029 min), and

4-linked GlcNAc (time 25.571 min).

The primary structures of two of the major oligosaccharides in CA215 is therefore tentatively assigned as:

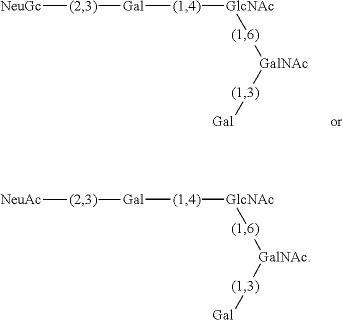

Additional structures are assigned as follows:

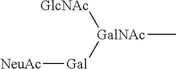

which has the structure

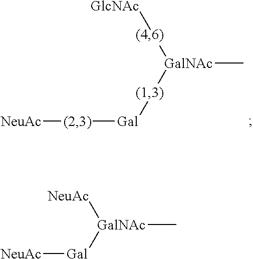

which has the structure

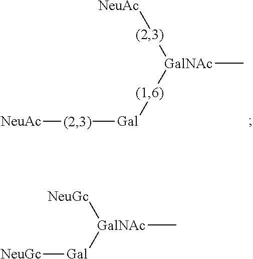

which has the structure

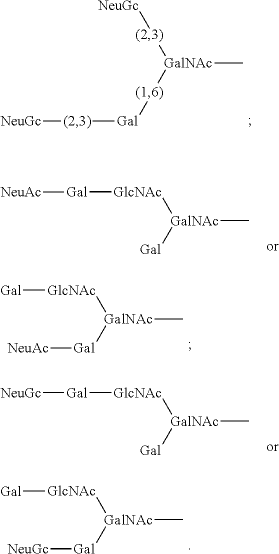

These structural assignments are confirmed by total synthesis of the glycans.

### Example 4

### Comparison of O-Linked Glycans from Selected Glycoprotein or Glycolipids

TABLE 2
Comparisons of O-linked Glycans from
various Glycoproteins or Glycolipids

| No. | Name | O-linked Glycans |
|---|---|---|
| 1. | Global H | Fuc-Gal-GalNAc-Gal-Gal-Glc-OH |
| 2. | SSEA-3 | Gal-GalNAc-Gal-Gal-Glc-OH |
| 3. | Mucin-1 | GalNAc—OH |
| | | Gal-GalNAc—OH |
| | | NeuAc-Gal-GalNAc—OH |
| 4. | CA125 | Gal-GlcNAc-Gal(Gal-GlcNAc—)-GalNAc—OH |
| | | Gal(Gal-GlcNAc—)-GalNAc—OH |
| 5. | IgA | GalNAc—OH |
| | | Gal-GalNAc—OH |
| 6. | IgD | NeuNAc(NeuAc-Gal-)-GalNAc—OH |
| 7. | CA215 | NeuGc-Gal-GalNAc—OH |
| | (disclosed | NeuAc(GlcNAc—)GalNAc—OH |
| | in this | NeuAc-Gal (GlcNAc—)-GalNAc—OH |
| | application) | NeuAc-Gal (NeuNAc—)-GalNAc—OH |
| | | NeuGc-Gal (NeuGc-)-GalNAc—OH |
| | | Gal (NeuAc-Gal-GlcNAc)-GalNAc—OH |
| | | Gal (NeuGlc-Gal-GlcNAc)-GalNAc—OH |
| | | NeuAc-Gal (Gal-GlcNAc)-GalNAc—OH |
| | | NeuGc-Gal (Gal-GlcNAc)-GalNAc—OH |

### Example 5

### Commonality of the Glycan Epitope

### Binding Between RP215 and CA215 Antigen Derived from Different Cancer Cells

The dissociation constants of antibody RP215 with respect to the CA215 antigen expressed on the surface of cancer cells C33A, ME180, OC-3-VGH and shed medium of OC-3-VGH cells were determined.

Enzyme-linked immunosorbent assays (ELISA) were performed to assess the relative binding of RP215 to well-coated CA215 obtained from these different cancer cell lines (ovarian or cervical) or in affinity purified form. Briefly, 1×10⁴ cultured cancer cells were coated and dried separately on microtiter wells, followed by blocking and washing. Similarly, CA215 affinity-purified from the shed medium of cultured OC-3-VGH cancer cells, was also coated on microwells for comparison. Standard ELISA were performed and dose-dependent binding of RP215 and well-coated CA215 were plotted against absorbance at 405 nm. The dissociation constants were estimated for each pair of binding assays. In all cases, the affinity constant was approximately 2-4×10⁻⁹M. The substantial identity of the dissociation constants supports the determination that the epitope is an O-linked glycan, since the immunoglobulin portions to which the O-type glycans are bound in these three different cell lines have different structures.

### Example 6

### Structural Analysis of N-Linked Oligosaccharides

Release of N-linked glycans: All samples were first dissolved with 1 mL nanopure H₂O followed by a freeze-drying step. The dried samples were then dissolved with 100 μL ammonium bicarbonate buffer (50 mM, pH 8.4) and followed immediately by reduction with 25 mM dithiothreitol (45 min at 50° C.) and carboxyamidomethylation with 90 mM iodoacetamide (45 min at room temperature in the dark) prior to trypsin digestion (37° C., overnight). A second enzyme, peptide N-glycosidase F (New England Biolabs) was added to each of the tryptic digests and incubated at 37° C. for 18 hours to release the N-linked glycans. After enzyme digestions, the samples were passed through a C18 reversed phase cartridge. The N-linked glycans from each sample were eluted with 5% acetic acid and lyophilized thereafter.

Preparation of the per-O-methylated carbohydrates: The lyophilized N-linked fraction of each sample was dissolved in dimethyl sulfoxide and then methylated for glycan structural analysis by mass spectrometry.

Matrix-assisted laser desorption/ionization time of flight mass spectrometry (MALDI-TOF MS): Initially, N-linked glycans were analyzed by MALDI-TOF MS using a Voyager V-DE™ mass spectrometer (Applied Biosystems). However, glycans could not be detected in any of the samples. Hence, the samples were analyzed by electrospray ionization mass spectrometry (ESI-MS) using an LCQ™-MS (Thermo Finnigan™) quadruple ion trap. Each sample (˜5 pmole/μL) was infused directly into the instrument at a constant flow rate of 1 μL/min via a syringe pump (Harvard Apparatus) and sprayed at 3.5 KV. A normal collision energy of 35 and an isolation mass window of 2 Da were applied to obtain MSn.

Structural analysis of N-linked oligosaccharides: Samples containing human IgG, RP215 and CA215 were analyzed separately by ESI-MS. The profiles of N-linked glycans of each immunoglobulin species were generated as described above and compared. The structures that are unique to CA215 and not in human IgG or RP215 were identified and are listed in Table 3 which indicates observed mass, charge state and proposed structures. As shown in this table, CA215 contains a mixture of high mannose structure as well as N-glycolylneuraminic acid (NeuGc) in the terminal bisecting N-glycan structure. In the case of human IgG, only N-acetylneuraminic acid was found.

TABLE 3
Profile of N-linked glycans unique to CA215

| Observed Mass | | |
|---|---|---|
| (M + Na) | Charge State | Proposed Structure |
| 1169 | Double | GlcNAc₅Man₃Hex₂ |
| 1172 | Single | GlcNAc₂Man₃ |
| 1330 | Double | GlcNAc₄Man₃Hex₂Fuc₁NeuGc₁ |
| 1366 ^{a} | Double | GlcNAc₅Man₅Hex₂NeuGc₁ |
| 1438 ^{a} | Double | GlcNAc₄Man₃Hex₂NeuGc₂ |
| 1467 ^{a} | Double | GlcNAc₅Man₅Hex₅NeuGc₁ |
| 1498 | Double | GlcNAc₆Man₃Hex₄ |
| 1525 ^{a} | Double | GlcNAc₄Man₃Hex₂Fuc₁NeuGc₂ |
| 1580 | Single | GlcNAc₂Man₅ |
| 1621 | Single | GlcNAc₃Man₃Hex₁ |
| 1785 ^{b} | Single | GlcNAc₂Man₆ |

^{a} OM with NeuGc as the terminal sialic acid.
^{b} OM with high mannose structure.

### Example 7

### Homology Analysis of Tryptic Peptides Derived from CA215

Methods for preparation of tryptic peptides of affinity purified CA215 were described by Lee, G., et al., Cancer Biol. & Ther. (2008) 7:2007-2014.

Following tryptic digestions of CA215, a total of 124 tryptic peptides were detected by MALDI-TOF MS. A Protein BLAST Service was conducted to identify proteins with a high degree of peptide sequence homology. The results of such comprehensive analysis are summarized in Table 4 according to the molecular nature of these identified proteins. Among these tryptic peptides detected from CA215, as many as 60% can be matched to IgSF proteins. These include antigen receptors (˜47.6%), antigen presenting MHC molecules (˜4.9%), cell adhesion molecules (˜8.1%), cytokine and growth factors (˜6.5%), receptor tyrosine kinase/phosphatase (˜5.7%), and others such as titin (˜9.7%). Another major category of these tryptic peptides which are not IgSF protein-related is mucins (˜7.3%).

TABLE 4
Homology Analysis of CA215 Based on MALDI-
TOF MS Analysis of Tryptic Peptides

| | | Number of Peptides Matched |
|---|---|---|
| Molecule Function/Category | | Total = 124 (Percentage) |
| I. | Antigen receptors | |
| | 1. Antibodies and immunoglobulins | 52 (42.0%) |
| | 2. T cell receptor chains | 7 (5.7%) |
| II. | Antigen presenting molecules | 6 (4.9%) |
| | (MHC I and MHC II) | |
| III. | Adhesion molecules | 10 (8.1%) |
| IV. | Cytokine and growth factors | 8 (6.5%) |
| V. | Receptor tyrosine kinase/phosphatase | 7 (5.7%) |
| VI. | Others | |
| | 1. IgSF related | 12 (9.7%) |
| | (e.g., titin) | Total with homology*: |
| | | 75/124 (60.5%) |
| | 2. IgSF unrelated | 9 (7.3%) |
| | (e.g., mucin) | |

*Excluding overlapping matched peptides

### Example 8

### Mapping of Glycosylation Sites

¹⁸O-labeling of CA215 was performed for N-glycosylation site mapping. O-Elimination followed by Michael addition (BEMAD) was used for O-glycosylation site mapping of the same CA215 sample. The ¹⁸O-labeled peptides were subsequently analyzed by LC-MS/MS (liquid chromatography with mass spectrometry/mass spectrometry). The peptide sequences as well as the potential N-glycosylation and O-glycosylation sites were determined. Protein BLAST Service was employed to identify those proteins which have the highest homology as well as the potential O- and N-linked glycosylation sites along the peptide sequences.

The most notable N-linked and O-linked glycopeptides with potential glycosylation sites were identified and listed in Table 5. A total of two N-linked and eight O-linked glycopeptides were detected through site mapping analysis. These ten glycopeptides with accession numbers of the originating proteins and potential glycosylation sites are listed in Table 5. Through NCBI Protein BLAST Services, known proteins with high peptide sequence homology are also listed.

TABLE 5
N-linked and O-linked glycosylation site mappings of CA215

| Accession | | Peptide Sequence Homology |
|---|---|---|
| Number | Peptide Detected^{a} | of Proteins (%) |
| I. | 1. EEQFNSTFR | Immunoglobulin heavy chain (Fc) |
| CAC12842.1 | | (100%) |
| II. | 2. EEQFNSTYR | Immunoglobulin heavy chain (Fc) |
| CAA04843.1 | | (100%) |
| III. | 3. LSVPTSEWQR | Cathepsin S (100%) |
| AAB60643.2 | | |
| IV. | 4. FTCLATNDAGDSSK | Hemicentin (100%) Titin (100%) |
| AAK68690.1 | | Palladin isoform 4 (92%) LRN4 |
| | | (78%) (IgSF proteins) |
| V. | 5. DTLMISR | Immunoglobulin heavy chain (Fc) |
| AAD38158.1 | | (100%) |
| VI. | 6. GYLPEPVTVTWNSGTLTNGVR | Immunoglobulin heavy chain (Fab) |
| AAC39746.2 | | (90%) |
| VII. | 7. SVSLTCMINGFYPSDISVEWEK | Immunoglobulin heavy chain (Fc) |
| AAN76042.1 | | (90%) |
| VIII. | 8. QSSGLYSLSSVVSVTSSSQPVTCNV | Immunoglobulin heavy chain (Fab |
| CAJ75462.1 | | and Fc) (100%) |
| IX. | 9. VYTMGPPREELSSR | Immunoglobulin heavy chain (Fc) |
| ABY48864.2 | | (98%) IgA variable region (89%) |
| | | IgM (98%) |
| X. | 10. TFPSVR | Zinc finger protein 414 isoform I |
| NP_001139647.1 | | (100%) Forkhead box protein C2 |
| | | (100%) Immunoglobulin heavy |
| | | chain variable region (83%) |

^{a}Bold letters indicate glycosylation sites
Fc: constant region of immunoglobulins
Fab: variable region of immunoglobulins
Lot CA215C (urea-eluted) was used for this analysis
Source Protein BLAST service: located on the World Wide Web at blast.ncbi.nlm.nih.gov/Blast.cgi

The two peptides with potential N-glycosylation sites (bold letter), EEQFNSTFR (CAC 12842.1) and EEQFNSTYR (CAA04843.1), were shown to match 100% with those of immunoglobulin heavy chains mostly in the constant region. One of the O-linked glycopeptides, LSVPTSEQWR (sites with bold letter) was found to match 100% with that of a lysosomal protease, cathepsin S. The other O-linked glycopeptide, FTCLATNDAGDSK (sites with bold letter) was found to match 100% with those of IgSF proteins including hemicentin, titin as well as several others. Unexpectedly, the remaining six O-linked glycopeptides were highly matched mostly to the variable Fab or Fc domains of immunoglobulin heavy chains.

### Example 9

### Location of the Carbohydrate Portion of the Epitope in the CA215 Immunoglobulin Region

Sandwich EIA were performed for the quantification of CA215 with RP215 Mab coated microwells in the presence of different enzyme-labeled antibody probes (Lee, et al. (2008), supra). Three different enzyme-labeled antibody probes, HRP-labeled RP215, ALP-labeled goat anti-human IgG Fc and ALP-labeled goat anti-human Fab were used separately for CA215 assays with one step 2 hr incubation at 37° C. The remaining steps for wash and subsequent color developments with substrate incubations were described previously (Lee, et al. (2008), supra).

Both enzyme-labeled RP215 and goat anti-human IgG Fc gave rise to good dose-dependent signals to sandwiched CA215. In contrast, enzyme-labeled goat anti-human IgG Fab did not pair with well-coated RP215 in the presence of CA215, under the same assay conditions. It thus appears the epitope resides predominantly in the Fab region of the CA215 immunoglobulin chain.

1. A compound selected from the group consisting of
GalNAc₁GlcNAc₁Gal₁NeuAc₁;
GalNAc₁Gal₁NeuAc₂;
GalNAc₁Gal₁NeuGc₂;
GalNAc₁GlcNAc₁Gal₂NeuAc₁; and
GalNAc₁GlcNAc₁Gal₂NeuGc.

2. The compound of claim 1 which is of the formula
GalNAc₁GlcNAc₁Gal₁NeuAc₁
and has the structure

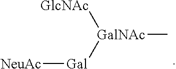

3. The compound of claim 2 which has the structure

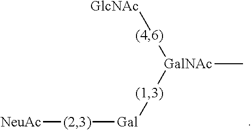

4. The compound of claim 1 which is of the formula
GalNAc₁Gal₁NeuAc₂
and has the structure

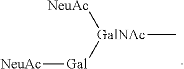

5. The compound of claim 4 which has the structure

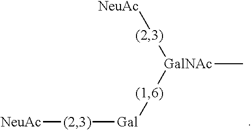

6. The compound of claim 1 which is of the formula
GalNAc₁Gal₁NeuGc₂
and has the structure

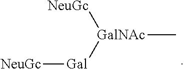

7. The compound of claim 6 which has the structure

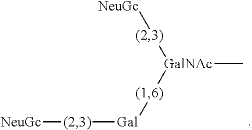

8. The compound of claim 1 which is of the formula
GalNAc₁GlcNAc₁Gal₂NeuAc₁
and has the structure

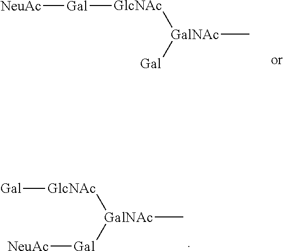

9. The compound of claim 8 which has the structure

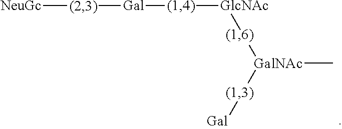

10. The compound of claim 1 which is of the formula
GalNAc₁GlcNAc₁Gal₂NeuGc
and has the structure

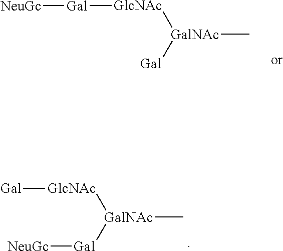

11. The compound of claim 10 which has the structure

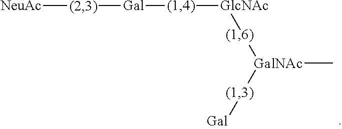

12. An immunogenic composition that contains as part of an immunogen at least one hapten consisting of a compound set forth in claim 1
wherein said hapten is coupled to a heterologous protein and/or wherein the composition contains an adjuvant.

13. (canceled)

14. An immunogenic composition that contains at least one of the compounds set forth in claim 1 in combination with at least one compound selected from the group consisting of
GlcNAc₅Man₃Hex₂;
GlcNAc₂Man₃;
GlcNAc₄Man₃Hex₂Fuc₁NeuGc₁;
GlcNAc₅Man₃Hex₂NeuGc₁;
GlcNAc₄Man₃Hex₂NeuGc₂;
GlcNAc₅Man₃Hex₃NeuGc₁;
GlcNAc₆Man₃Hex₄;
GlcNAc₄Man₃Hex₂Fuc₁NeuGc₂;
GlcNAc₂Man₅;
GlcNAc₃Man₃Hex₁; and
GlcNAc₂Man₆.

15-16. (canceled)

17. The composition of claim 12 wherein said compounds are coupled to positions corresponding to glycosylation sites present in an Fab region.

18. A method to induce an immune response directed to a tumor in a subject bearing a cancer that expresses an epitope that comprises a moiety selected from the group consisting of
GalNAc₁GlcNAc₁Gal₁NeuAc₁;
GalNAc₁Gal₁NeuAc₂;
GalNAc₁Gal₁NeuGc₂;
GalNAc₁GlcNAc₁Gal₂NeuAc₁; and
GalNAc₁GlcNAc₁Gal₂NeuGc
which method comprises administering to said subject an effective amount of the compound of claim 1.

19. A method to induce an immune response directed to a tumor in a subject bearing a cancer that expresses an epitope that comprises a moiety selected from the group consisting of
GalNAc₁GlcNAc₁Gal₁NeuAc₁;
GalNAc₁Gal₁NeuAc₂;
GalNAc₁Gal₁NeuGc₂;
GalNAc₁GlcNAc₁Gal₂NeuAc₁; and
GalNAc₁GlcNAc₁Gal₂NeuGc
which method comprises administering to said subject an effective amount of the composition of claim 14.

20. A method to prepare antibodies immunoreactive with an epitope that comprises a moiety selected from the group consisting of
GalNAc₁GlcNAc₁Gal₁NeuAc₁;
GalNAc₁Gal₁NeuAc₂;
GalNAc₁Gal₁NeuGc₂;
GalNAc₁GlcNAc₁Gal₂NeuAc₁; and
GalNAc₁GlcNAc₁Gal₂NeuGc
which method comprises administering to a subject the composition of claim 12, and recovering antibodies from said subject.

21. The method of claim 20 which further comprises recovering antibody-producing cells from said subject and isolating antibodies or nucleic acid encoding said antibodies from said cells.

22. Antibodies prepared by the method of claim 21.

23-27. (canceled)

28. The composition of claim 14 wherein said compounds are coupled to positions corresponding to glycosylation sites present in said Fab region.
